(19) United States
(12) Patent Application Publication
(10) Pub. No.: US 2023/0268042 A1
(43) Pub. Date: Aug. 24, 2023

(54) BLOCKCHAIN BASED HEALTHCARE MANAGEMENT NETWORK

(71) Applicants: US TECHNOLOGY INTERNATIONAL PRIVATE LIMITED, Thiruvananthapuram, Kerala (IN); JOTHYDEV'S DIABETES RESEARCH CENTRE, Trivandrum, Kerala (IN)

(72) Inventors: Jaison Joseph SEBASTIAN, Trivandrum (IN); Jothydev KESAVADEV, Trivandrum (IN); Pradeep JOSEPH, Trivandrum (IN); Vinu SREEDHARAN, Trivandrum (IN); Sambhu Prasad Balakrishna PILLAI, Trivandrum (IN)

(21) Appl. No.: 18/024,161

(22) PCT Filed: Sep. 1, 2021
(86) PCT No.: PCT/IN2021/050843

§ 371 Date: Mar. 1, 2023

(30) Foreign Application Priority Data
Sep. 1, 2020 (IN) 202041037756

(51) Int. Cl. G16H 20/00; G16H 10/60; G16H 50/70

(52) U.S. Cl. CPC G16H 20/00; G16H 10/60; G16H 50/70

(57) ABSTRACT

The present subject matter refers to a method for optimized utilization of resources in the blockchain based healthcare network comprising acquiring patient data from a plurality of device nodes and provider nodes associated with a plurality of patients and computing an adherence score for each patient in a corresponding patient cluster of the plurality of patient clusters based on the EHR and the vital information of a corresponding patient included in corresponding patient cluster of the plurality of patient clusters. The method further comprises distributing the computed adherence score to payor nodes in the blockchain based healthcare network and receiving wellness points corresponding to each patient of the plurality of patients from the payor nodes in response to the distributed adherence score. Furthermore, the method comprises allocating the received wellness points to a respective device node associated with a corresponding patient of the plurality of patients.

### FIELD OF THE INVENTION

The present disclosure generally relates to a method and a system in a blockchain based healthcare network, and, more particularly, to a method and a system that implements a blockchain technique and provides optimized utilization of resources and increases the effectiveness of resources in the blockchain based healthcare network.

### BACKGROUND OF THE INVENTION

In recent years, the healthcare network has become complex over years with a host of entities—for example payors, various types of providers viz primary care providers, secondary care providers, tertiary care providers, the actors within the provider ecosystem, the pharmacy companies, the laboratories, research organizations, app developers, retailers, transportation companies, family, friends, peers who have the same disease, local community organizations to state a few. These entities and their actors should work in cohesion with the patient and their care group, to provide favorable outcomes to the patient in an effective manner. For example, if a patient with diabetes takes diabetes care, the major entities in such case are the payor, the physicians, the care coordinators, the peer coaches across the provider levels, the pharmacy, the app developers, the patients, and their care group. A diabetes patient cannot keep diabetes in control, just by the physician prescribing the tests and drugs, especially a lifestyle disease like diabetes which typically extends through the lifetime of a patient.

There is a need for the peer coach to prescribe the diet and fitness routine keeping in perspective the nuances of the patient, for example, but not limited to whether the patient fasts during the lent season or Ramadan season. There is also a need for the care coordinator to follow up with the patients to ensure that they take the tests, take the drugs as prescribed and follow the diet and fitness routine as instructed. Essentially these actors across the care levels form a network that serves the patient.

In order to serve the patient in an effective manner to achieve the single most objective which is to keep the diabetes in control, following leading indicators needs to be analyzed. Firstly, is the patient adhering to the tests that have been prescribed by the physician. Secondly, is the patient adhering to the drugs that have been prescribed after the physician has looked at the test results. Thirdly, is the patient adhering to the lifestyle viz the diet and fitness routine instructed by the peer coach. In recent healthcare technologies it is determined by the actors in the healthcare network based on a questionnaire method, responses of the questionnaire method, and other parameters like tracking the number of steps walked by the patient in a day or IoT based pill reminders in the case of drugs. In the questionnaire method, asking questions to the patient to assess their adherence levels, a provider is forced to trust the patient which might work well for a patient or two. but not at scale when it comes to management of the health of a population. In the case of counting the steps, the challenge is the provider does not know if the patient at the end of the day feasted on food with high sugar content or took the food in the order the patient was expected to. In the case of IoT based pill reminders, while the provider will know when the patient has taken the drug from the pill reminder, but the provider may not know for sure if the patient has taken the drug.

For a patient to have the right outcomes at a right time, firstly it is required that a regulator or an insurance company, or a provider can diagnose the network digitally. Secondly, there is also a requirement that the basis for the diagnosis of the network has to be made available for audit, for clinical research, to the right stakeholders for future decision making, and to ensure there are no disparities in the utilization of resources.

Therefore, there lies a need for a method and a system that can provide an interface to the regulator or the insurance company, or the provider to diagnose the network digitally. Further, there is also a need for a method and a system that is capable of digitally mapping the right care actors across the network to the right patients based on their demographic, clinical, and behavioral parameters such that the resources are optimally utilized for the right patients. Furthermore, there lies a need for a method and a system that can avail the basis for the diagnosis of the network for the audit and the clinical research to the right stakeholders for future decision making such that there are no disparities in the utilization of the resources.

### SUMMARY OF THE INVENTION

This summary is provided to introduce a selection of concepts, in a simplified format, that are further described in the detailed description of the invention. This summary is neither intended to identify key or essential inventive concepts of the invention and nor is it intended for determining the scope of the invention.

Aspects of the present disclosure provides a method and a system for optimized utilization of resources for optimizing the role of various players and increasing the effectiveness of various players in the blockchain based healthcare network.

In an implementation, the present subject matter refers to a method for optimized utilization of resources in the blockchain based healthcare network. The method comprises acquiring patient data from a plurality of device nodes and provider nodes associated with a plurality of patients, wherein the patient data includes Electronic Health Record (EHR) and vital information of each of the plurality of patients. The method further comprises computing an adherence score associated with at least one Key Performance Indicator (KPI) for each patient in a corresponding patient cluster of the plurality of patient clusters based on the EHR and the vital information of a corresponding patient included in corresponding patient cluster of the plurality of patient clusters. Furthermore, the method comprises distributing the computed adherence score to payor nodes in the blockchain based healthcare network and receiving wellness points corresponding to each patient of the plurality of patients from the payor nodes in response to the distributed adherence score. The wellness points are generated by the payor nodes based on the computed adherence score. After receiving the wellness points, the method thereafter comprises allocating the received wellness points to a respective device node and provider nodes associated with a corresponding patient of the plurality of patients.

In another implementation, the present subject matter refers to a system for optimized utilization of resources in the blockchain based healthcare network. The system includes a plurality of device nodes and provider nodes associated with a plurality of patients. The plurality of device nodes and the provider nodes generates patient data that includes Electronic Health Record (EHR) and vital information of each of the plurality of patients. The system further includes a plurality of payor nodes and a management server. The management server acquires the patient data from the plurality of device nodes and the provider nodes associated with the plurality of patients and clusters the plurality of patients into a plurality of patient clusters based on the patient data including the EHR, demographic parameters, behavioral parameters, and the vital information of each of the plurality of patients. Further, the management server computes an adherence score associated with at least one Key Performance Indicator (KPI) for each patient in a corresponding patient cluster of the plurality of patient clusters based on the EHR and the vital information of a corresponding patient included in corresponding patient cluster of the plurality of patient clusters, and distributes the computed adherence score to the provider nodes and the plurality of payor nodes. The plurality of payor nodes generates wellness points corresponding to each patient of the plurality of patients based on the computed adherence score and transmits the generated wellness points to the management server in response to the distributed adherence score. Thereafter, the management server receives the wellness points from the plurality of payor nodes and allocates the received wellness points to a respective device node and provider nodes associated with a corresponding patient of the plurality of patients.

According to an implementation of the present disclosure, a plurality of patients is clustered into a plurality of patient clusters based on clinical parameters, demographic parameters, and behavior parameters. Subsequently, a test adherence score, a drug adherence score, a lifestyle adherence score, and diabetes in control score for each patient in a given cluster is calculated based on various computation techniques. Each of the computation techniques described in the present disclosure involves digital computation and does not involve user's questionnaire responses or user's feeds. Every transaction that computes the adherence scores is written into the blockchain. Furthermore, based on the aforementioned scores, wellness points are allocated to the patient device nodes and which is also written into the blockchain. The corresponding vitals and algorithm that computes the scores are hashed and written into the blockchain so as to facilitate digital audits at a click of a button.

In an example, the patient may use the wellness points for performing availing services from one or more providers, including, but not limited to, care provider, app developer, retailer, pharmacy, etc. Such an availing of services may be recorded as blockchain transactions and may be maintained uniformly across all the parties to the blockchain network. An access control list that is hashed and stored in the blockchain governs who has access to what kind of patient information ensuring the data privacy. The access control list can be managed by the patient directly and the associated transaction is stored in blockchain.

According to another implementation of the present disclosure, a healthcare platform is also provided. The healthcare platform provides a centralized avenue for viewing, updating, and analyzing data pertaining to multiple players involved in a health care network system. For example, the progression of diabetes stage from Insulin resistance to Prediabetes to Type 2 Diabetes, to Type 2 Diabetes with complications is either stalled or the velocity is reduced, wherein the physician can debug a patient digitally and the regulator can diagnose the healthcare network that serves the patient clusters.

To further clarify the advantages and features of the present invention, a more particular description of the invention will be rendered by reference to specific embodiments thereof, which is illustrated in the appended drawing. It is appreciated that these drawings depict only typical embodiments of the invention and are therefore not to be considered limiting its scope. The invention will be described and explained with additional specificity and detail with the accompanying drawings.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1A illustrates an example of a health care network, according to an embodiment of the present disclosure;

FIG. 1B illustrates a graphical example of a health care network, according to an embodiment of the present disclosure;

FIG. 2 illustrates an overview of a blockchain based healthcare system environment, according to an embodiment of the present disclosure;

FIG. 3 illustrates a block diagram of a blockchain based healthcare system, according to an embodiment of the present disclosure;

FIG. 4 illustrates an example overview of clustering process to cluster patients into clusters, according to an embodiment of the present disclosure;

FIG. 5 illustrates a block diagram of adherence score computation modules of the management server of FIGS. 2 and 3, according to an embodiment of the present disclosure;

FIG. 6 illustrates an implementation of a test adherence computation technique for determining a level of adherence to tests for one or more patients, according to an embodiment of the present disclosure;

FIG. 7A illustrates an implementation of a drug adherence computation technique for determining a level of adherence to drugs for one or more patients, according to an embodiment of the present disclosure;

FIG. 7B illustrates an implementation of a lifestyle adherence computation technique for determining a level of adherence to lifestyle for one or more patients, according to an embodiment of the present disclosure;

FIG. 8 illustrates an implementation of a syndrome specific adherence computation technique for determining a syndrome specific control level for one or more patients, according to an embodiment of the present disclosure;

FIG. 9 illustrates an example blockchain network implementing a management server, a controller, a payor node, a provider node, a patient node including an Adherence algorithm processing (AAP) Device, and technology provider and payor system, according to an embodiment of the present disclosure;

FIG. 10 illustrates a graphical user interface provided by patient debug device of the blockchain based healthcare system, according to an embodiment of the present disclosure;

FIG. 11 illustrates an example implementation of generation of hashes by the AAP device and interaction with a blockchain network, according to an embodiment of the present disclosure;

FIG. 12 illustrates interactions between a patient node and a blockchain network, according to an embodiment of the present disclosure;

FIG. 13 illustrates a graphical user interface of a blockboard in a healthcare network platform, according to an embodiment of the present disclosure;

FIG. 14 illustrates a flow chart of method steps performed by a management server in the blockchain based healthcare network, according to an embodiment of the present disclosure; and

FIG. 15 illustrates an example of a graphical user interface in a form of the blockboard, according to another embodiment of the present disclosure.

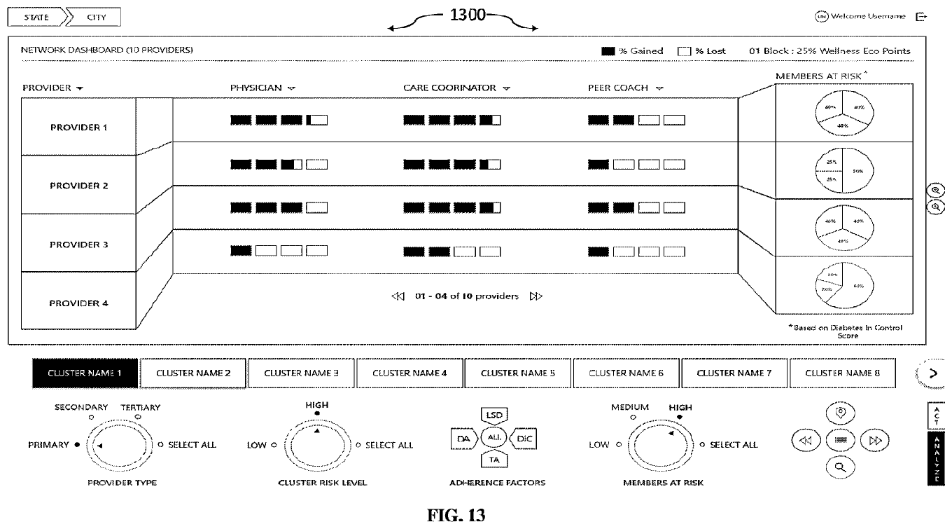

Further, it is to be appreciated that a computer screen layout of a graphical user interface used for diagnosing healthcare networks corresponding to the FIG. 13 is being protected under the WIPO Hague system as a design application with a reference application number WIPO98059.

Furthermore, skilled artisans will appreciate that elements in the drawings are illustrated for simplicity and may not have necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having the benefit of the description herein.

### DETAILED DESCRIPTION OF THE INVENTION

It should be understood at the outset that although illustrative implementations of the embodiments of the present disclosure are illustrated below, the present invention may be implemented using any number of techniques, whether currently known or in existence. The present disclosure should in no way be limited to the illustrative implementations, drawings, and techniques illustrated below, including the exemplary design and implementation illustrated and described herein, but may be modified within the scope of the appended claims along with their full scope of equivalents.

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to no embodiments or to one embodiment or to several embodiments or to all embodiments. Accordingly, the term “some embodiments” is defined as meaning “no embodiment, or one embodiment, or more than one embodiment, or all embodiments.”

The terminology and structure employed herein is for describing, teaching, and illuminating some embodiments and their specific features and elements and does not limit, restrict, or reduce the spirit and scope of the claims or their equivalents.

More specifically, any terms used herein such as but not limited to “includes,” “comprises,” “has,” “consists,” and grammatical variants thereof do NOT specify an exact limitation or restriction and certainly do NOT exclude the possible addition of one or more features or elements, unless otherwise stated, and furthermore must NOT be taken to exclude the possible removal of one or more of the listed features and elements, unless otherwise stated with the limiting language “MUST comprise” or “NEEDS TO include.”

Whether or not a certain feature or element was limited to being used only once, either way, it may still be referred to as “one or more features” or “one or more elements” or “at least one feature” or “at least one element.” Furthermore, the use of the terms “one or more” or “at least one” feature or element do NOT preclude there being none of that feature or element, unless otherwise specified by limiting language such as “there NEEDS to be one or more . . . ” or “one or more element is REQUIRED.”

Unless otherwise defined, all terms, and especially any technical and/or scientific terms, used herein may be taken to have the same meaning as commonly understood by one having ordinary skill in the art. The system, methods, and examples provided herein are illustrative only and not intended to be limiting.

Embodiments of the present invention will be described below in detail in a non-limiting manner with reference to the accompanying drawings.

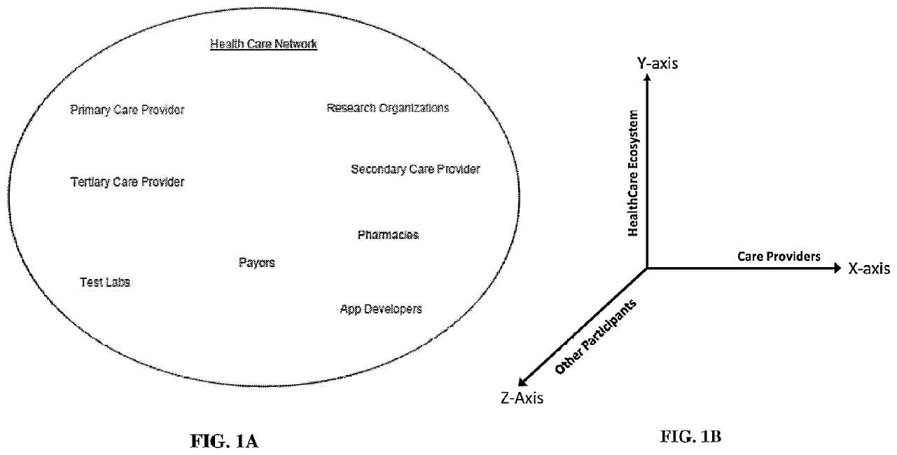

FIG. 1A illustrates an example of a health care network, according to an embodiment of the present disclosure. The healthcare network includes, but not limited to, payors, various types of providers viz primary care providers, secondary care providers, tertiary care providers, the actors within the provider ecosystem, the pharmacy companies, the laboratories, research organizations, app developers, retailers, transportation companies, family, friends, peers who have the same disease, local community organizations.

Referring now to FIG. 1B, FIG. 1B a graphical example of a health care network, according to an embodiment of the present disclosure. X-axis of FIG. 1B represents a network of care providers. The care providers network may include, but not limited to, the primary care providers, the secondary care providers, the tertiary care providers. Y-axis of FIG. 1B represents a network of healthcare ecosystem. The healthcare ecosystem may include the laboratories, research organizations, local community organizations, and pharmacy companies. Z-axis of FIG. 1B represents other participants of healthcare network. The other participants may correspond to, but not limited to, the, app developers, retailers, transportation companies, care group, community kitchen that serve the patient, family, friends, peers who have the same disease etc . . . The other participants may also correspond to telecommunication companies or telco that serves as the intermediaries to digitally connect all of the ecosystem players in the healthcare network.

In an example, the healthcare network may be defined in a multi hierarchical modes (for example, Hierarchy A, Hierarchy B, and Hierarchy C). A first level under the Hierarchy A may corresponds to specialty (for example but not limited to Diabetes, Oncology) or programs (for example but not limited to smoking cessation, pregnancy). The second level under the Hierarchy A may include the patients in clinical clusters specific to the specialty. At the third level the patients may be categorized into high risk and low risk patients.

The Hierarchy B may correspond to a provider hierarchy, the provider hierarchy is a hierarchy where multiple levels corresponding to various care levels are present, for example but not limited to Primary Care, Secondary Care, Tertiary Care. Under each of the care levels, the providers are present. Under each of the providers, there are present provider actors, for example, but not limited to, the physician, peer coach, the care coordinator. Likewise, the Pharmacies are also provided under each care levels.

The Hierarchy C may correspond to a geographic hierarchy, multiple hierarchical levels based on how a country is organized. For example, country followed by a state followed by countries.

There could be other hierarchies that may be defined as the healthcare network expands, for example, but not limited to, inclusion of the clinical device companies, the care group comprising of family members, friends, community organizations. Accordingly, whenever a transaction is written into the blockchain the associated network hierarchy is passed on to the blockchain.

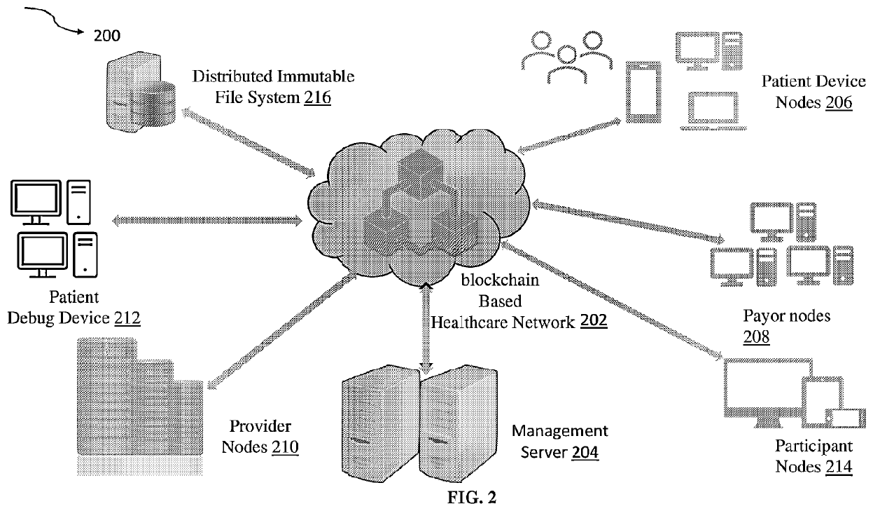

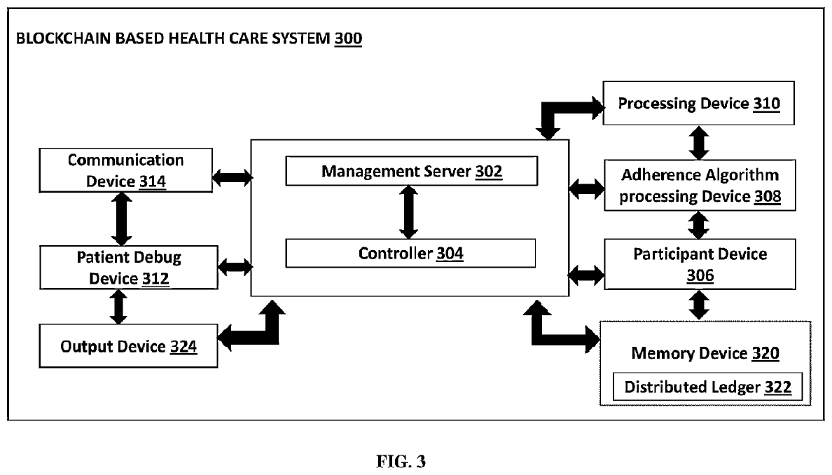

Now a detailed description of a blockchain based healthcare system will be explained with reference to FIGS. 2 and 3 of the drawings. FIG. 2 illustrates an overview of a blockchain based healthcare system environment 200, according to an embodiment of the present disclosure. As illustrated in FIG. 2, the Management Server 204 is operatively coupled, via a blockchain based healthcare network 202 to the Patient Device Nodes 206, Payor nodes 208, Provider Nodes 210, Patient Debug Device 212, Participant Nodes 214, and a distributed immutable file system 216. FIG. 2 illustrates only single example of an embodiment of the blockchain based healthcare system environment 200, and it will be appreciated that in other embodiments one or more of the systems, devices, or servers may be combined into a single system, device, or server, or be made up of multiple systems, devices, or servers.

The blockchain based healthcare network 202 may be a system specific distributive network receiving and distributing specific network feeds and identifying specific network associated triggers. The blockchain based healthcare network 202 may comprise a single, local network, a large network or a plurality of small or large networks interconnected together. The network 107 may comprise any type or a global area network (GAN), such as the Internet, a number of local area networks (LANs) broadband networks, wide area networks (WANs), etc. The blockchain based healthcare network 202 may include the Internet or incorporate portions thereof, such intranet(s), extranet(s), etc. Further, blockchain based healthcare network 202 may incorporate one or more LANs, wireless portions and may incorporate one or more of various protocols and architectures such as TCP/IP, Ethernet, ATM, etc. The blockchain based healthcare network 202 may also include other types of public networks, such the public switch telephone network (PSTN) or the like. The blockchain based healthcare network 202 may provide for wireline, wireless, or a combination wireline and wireless communication between devices on blockchain based healthcare network 202.

The Management Server 204 provides information to the patient device nodes 206, the Payor nodes 208, the Provider Nodes 210, the Patient Debug Device 212, the Participant Nodes 214, and the distributed immutable file system 216 and receives information from these nodes and devices in response to the provided information. Data obtained from several nodes can be stored in the distributed immutable file system 216. For example, the Management Server 204 can provide and manage a distributed ledger managed by a peer-to-peer network collectively adhering to a protocol for inter-node communication and validating new blocks. The Management Server 204 can also manage healthcare blockchain applications. The management server 204 may also include one or more processors to execute a set of instructions for managing the flow of information in the blockchain based healthcare network 202 and to control several nodes included in the the blockchain based healthcare network 202.

The patient device nodes 206 may correspond to various devices, such as wearables devices worn by patients, Internet of Things (IoT) enabled medical devices associated with the patients, patients a personal computer (PC), a tablet PC, a personal digital assistant (PDA), a mobile device, a palmtop computer, a laptop computer, a desktop computer, a communications device, a wireless telephone, a land-line telephone, a web appliance, a network router, switch or bridge, or any other machine capable of executing a set of instructions.

The Payor nodes 208 may corresponds to the claims systems and provider systems associated with the payors in the blockchain based healthcare network 202. The claim systems and provider systems are systems that connect with the patient device nodes 206 and the provider nodes 210 in the blockchain based healthcare system environment 200.

The provider nodes 210 may corresponds to system and devices of the providers and the actors within the provider ecosystem of the blockchain based healthcare system environment 200.

The Patient Debug Device 212 corresponds to a device managed by providers at a lower level in the within the provider ecosystem. The Patient Debug Device 212 may correspond to a Bluetooth enabled device.

The Participant Nodes 214 may correspond to devices and systems managed by other participants in the blockchain based healthcare system environment 200. For example, devices and systems managed by, but not limited to, the app developers, the retailers, the transportation companies, the care group, the community kitchen, family, friends, peers etc . . . , in the blockchain based healthcare system environment 200.

The distributed immutable file system 216 is a database that stores information regarding transactions and other information required for processing corresponding to the Management Server 204 and the several nodes (patient device nodes 206, Payor nodes 208, provider nodes 210, Patient Debug Device 212, and Participant Nodes 214) of the blockchain based healthcare system environment 200.

Referring now to FIG. 3, FIG. 3 illustrates a block diagram of a blockchain based healthcare system 300, according to an embodiment of the present disclosure. The blockchain based healthcare system 300 includes a Management Server 302, a Controller 304, Participant Device 306, Adherence algorithm processing (AAP) Device 308, Processing Device 310, a Patient Debug Device 312, a Communication Device 314, Memory Device 320 including a Distributed Ledger 322, and Output device 324.

The Management Server 302 corresponds to the Management Server 204 of the blockchain based healthcare system environment 200. The AAP Device 308 corresponds to the patient device nodes 206 of the blockchain based healthcare system environment 200. The

Processing Device 310 corresponds to the Payor nodes 208 and the provider nodes 210 of the blockchain based healthcare system environment 200. The Patient Debug Device 312 corresponds to the Patient Debug Device 212 of the blockchain based healthcare system environment 200.

The Controller 304 controls operations between the management server and also make operation specific decisions. The controller 304 may also controls individual components of the blockchain based healthcare system 300 based on information characteristics and processed information associated with the management server 302 and individual devices (the Participant Device 306, the AAP Device 308, the Processing Device 310, the Patient Debug Device 312, and the Communication Device 314).

The individual devices may include one or more processors and other support circuits and/or combinations of the foregoing. Control and signal processing functions of blockchain based healthcare system 300 are allocated between these individual devices according to their respective capabilities. The one or more processors may have the functionality to operate one or more software programs based on computer-readable instructions thereof, which may be stored in the memory device 320.

The individual devices are operatively coupled to the Communication Device 314 and the memory device 320. The individual devices use the Communication Device 314 to communicate with each other in the blockchain based healthcare network 202. As such, the communication device 314 generally comprises a server, or other device for communicating with the individual devices in the blockchain based healthcare network 202.

The memory device 320 stores, but is not limited to, a block chain application and the distributed ledger 322. In some embodiments, the distributed ledger 322 stores data including, but not limited to, at least portions of a transaction record. In one embodiment of the invention, both the block chain application and the distributed ledger 322 may associate with applications having computer-executable program code that instructs the individual devices the one or more processors to operate the communication device 314 to perform certain communication functions involving described herein. In one embodiment, the computer-executable program code of an application associated with the distributed ledger 322 and block chain application may also instruct the individual devices to perform certain logic functions, data processing, and data storing functions.

The Output device 324 may include a graphical user interface (GUI), and/or interfaces that may be configured to act as an output interface for the user's in the in the blockchain based healthcare network 202. The GUI may refer to a graphics display provided on a display (e.g., screen) of an electronic device. Other examples of the Output device may include graphics devices/display devices, Computer screens, alarm systems, smart phone display screens, dashboard mounted display screens in automobiles, or any other type of data output device. The output device may display information outputted by the individual devices on a display screen.

Now a detailed description will be made regarding operations of the devices and components included in the blockchain based healthcare system 300 with reference to the FIG. 2 and FIG. 3 of the drawings.

The patient device nodes 206 and the provider nodes 210 are associated with one or more patients and are configured to generate patient data that includes Electronic Health Record (EHR) and vital information of each of the one or more patients. The EHR may also include at least one of vital information, clinical information, demographic information, and behavior information of the plurality of patients.

The Management Server 204 or 302 acquires the patient data from the patient device nodes 206 and the provider nodes 210 associated with the plurality of patients and clusters the one or more patients into one or more patient clusters based on the patient data including the EHR, demographic parameters, behavioral parameters, and the vital information of each of the one or more patients.

The management server 204 or 302 further computes an adherence score associated with at least one Key Performance Indicator (KPI) for each patient in a corresponding patient cluster of the one or more patient clusters based on the EHR and the vital information of a corresponding patient included in the corresponding patient cluster of the one or more patient clusters. The KPI may correspond, but not limited to, an adherence factor. The KPI may correspond to factors other than the adherence within a healthcare environment. The computed adherence score corresponds to at least one of a test adherence score, a drug adherence score, a lifestyle adherence score, and a syndrome specific adherence score corresponding to each patient in each of the one or more patient clusters.

The management server 204 or 302 further distributes the computed adherence score to the provider nodes 210 and the payor nodes 208.

The payor nodes 208 receives the computed adherence score distributed by the management server 204 or 302 and generates wellness Eco points corresponding to each patient of the one or more patients based on the received computed adherence score. Thereafter, the payor nodes 208 transmits the generated wellness Eco points to the management server in response to the distributed adherence score.

Furthermore, the management server 204 or 302 further receives the wellness Eco points transmitted by the payor nodes 208 and allocates the received wellness Eco points to each of the provider nodes 210 and a respective patient device node among the patient device nodes 206 associated with a corresponding patient of the one or more patients. Further, the management server 204 or 302 may also distribute the allocated wellness Eco points of the respective device node to a respective participant node of the participant nodes 214 in the blockchain based healthcare system environment 200.

The management server 204 or 302 may also move at least one patient from one clinical cluster to other clinical cluster based on a progression of the wellness Eco points corresponding to the at least one patient.

The Patient Debug Device 212 or the Patient Debug Device 312 acquires one or more criteria of each of adherence parameters corresponding to the computed adherence score by the management server 204 or 302 and further provide a graphics interface including one or more adherence factors and a deviation chart including information regarding at least one of a positive deviation and a negative deviation of a corresponding patient of the one or more patients against each of the one or more criteria with respect to each of a baseline value and a threshold value for each of the one or more criteria. The graphics interface may also include, for example but not limited to, a cluster ID of corresponding patients among the one or more clusters, the adherence parameters, and a level of adherence of the patient to the adherence factors.

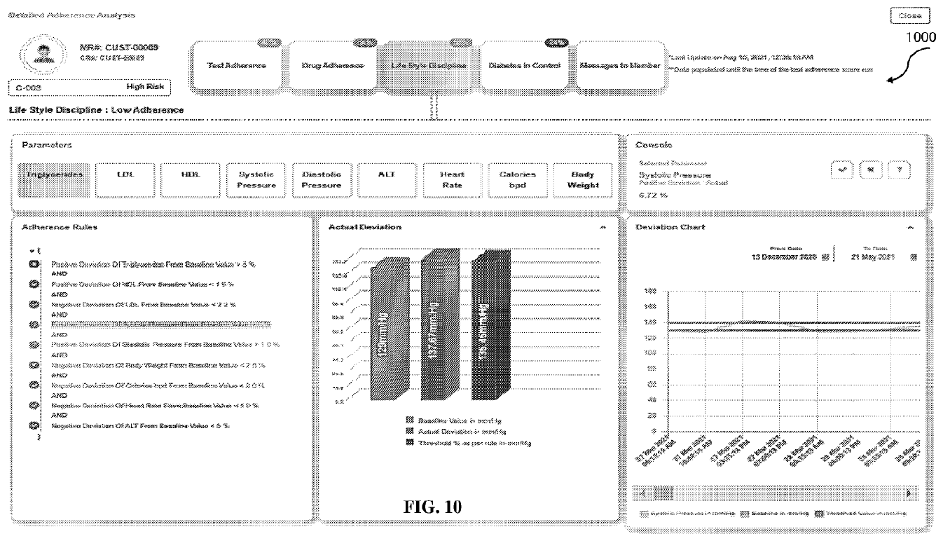

Once the graphics interface is provided by the patient debug device, a user of the patient debug device 212 or the patient debug device 312, for example but not limited to, a junior physician can debug a patient after selecting a particular adherence factor, for example lifestyle discipline. Further, the junior physician can also put a tick/cross/question mark against each of the adherence parameters used to arrive at a level of lifestyle discipline percentage, after looking the baseline value and the threshold value for each parameter. The Patient Debug Device 212 or the Patient Debug Device 312 receives inputs of the user, for example, the tick/cross/question mark against each of the adherence parameters and further generates an adherence snapshot of adherence rules based on the received input against each of the adherence parameters with reference to information included in the graphics interface. The generated adherence snapshot is auditable and editable based on the user's input. Further, the Patient Debug Device 312 also generates a pattern of the adherence snapshot as a function of time in response to corresponding inputs by the the junior physician against each of the adherence parameters. The generated adherence snapshot and the graphics interface of the patient debug device 212 or 312 is shown in FIG. 10 of the drawings. FIG. 10 illustrates a graphical user interface provided by the patient debug device 212 or 312 of the blockchain based healthcare system, according to an embodiment of the present disclosure. The patient debug device 212 or 312 provides the graphic user interface 1000. As shown in FIG. 10, the graphics user interface 1000 represent details of adherence analysis including particularly the adherence chart of the adherence rules, the adherence parameters, actual deviation chart, deviation chart, a console for receiving the user's input, a selection menu list of the adherence, the level of the adherence of the patient to the adherence factors.

The patient debug device 212 or 312 can further sync up automatically with patient identifiable tag. As an example, the patient debug device 212 or 312 may automatically display a patient record on the device with the crosses, question marks and ticks in an order for each of the adherence parameters.

The patient debug device 212 or 312 provides various advantages to the blockchain based healthcare system 300. For example, a main physician can look at only the cross marks and the question marks included in the generated adherence chart by the patient debug device 212 or 312. This reduces the time required from the main physician and that would help focus on specific points during patient consultation. This might also give hands-on experience to the junior physician to build expertise and also may serve as an auditable record and reference in future. Ultimately the patient debug device 212 or 312 helps bridge the gap in the physician to patient ratio and helps experienced physicians to focus on patients that need high level of care.

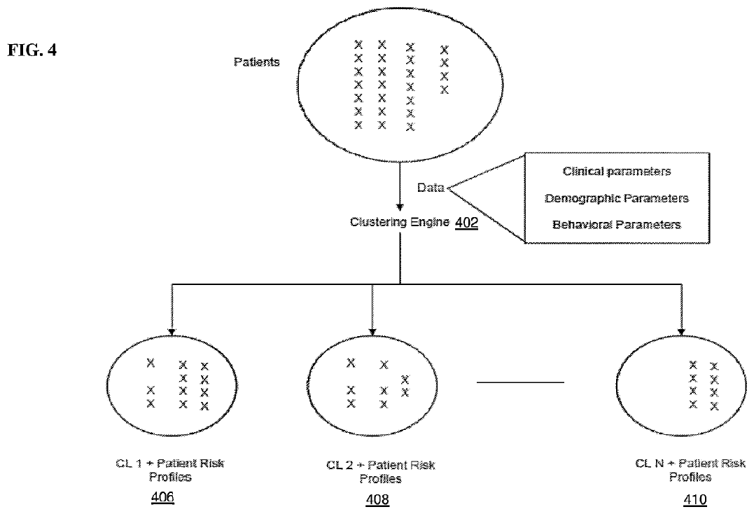

Now an example explanation of the clustering of the one or more patients into one or more patient clusters by the management server 204 or 302 will be made with reference to FIG. 4 of the drawings.

Referring to FIG. 4 of the drawings, FIG. 4 illustrates an example an example overview of a clustering process performed by the management server 204 or 302 to cluster the one or more patients into the one or more clusters, according to an embodiment of the present disclosure. The management server 204 or 302 includes a clustering engine 402. The clustering engine 402 receives the patient data from the patient device nodes 206 and the provider nodes 210 including the EHR, the demographic parameters, the behavioral parameters, and the vital information of each of the one or more patients and thereafter clusters the one or more patients into the one or more patient clusters based on the received patient data.

The management server 204 or 302 determines a risk level of patients included in the corresponding patient cluster of the one or more patient clusters based on the computed adherence score and categorizes the one or more patients within each of the one or more patient clusters based on the determined risk level of patients. As shown in FIG. 4 of the drawings, the patients are clustered into multiple clusters 406, 408, and 410 (i.e CL 1, CL 2, . . . , CL N) and are categorized by the management server 204 or 302 according to the determined risk level of the patients. As an example, diabetes patients as well as patients without diabetes are classified into clinical clusters identified by computation techniques driven based on the clinical, the demographic and the behavioral parameters. In each of the clinical clusters, the patients are categorized as one of a high risk and low risk. In an example implementation, the management server 204 or 302 determines, but not limited to, a risk level of the patients with diabetes based on the on the computed adherence score and categorizes the patients into predetermined RED, GREEN and YELLOW categories based on the determined risk level of the patients, where RED corresponds to patients with risk of the highest order in that clinical cluster.

According to an embodiment of the present disclosure, for each of the clinical cluster, a test adherence computation technique is provided to determine a level of adherence to tests, by the patients belonging to that clinical cluster. The level of the adherence to the tests, sets the foundation for clinical data that serves as a basis for the clinical cluster, drug adherence, lifestyle discipline adherence, and Syndrome Specific adherence (for example, diabetes in control).

To determine the level of drug adherence specific to the patients in each of the clinical clusters, key vitals corresponding to a plurality of drugs are analyzed using analytical and computational techniques to be described below. As an example, for each clinical cluster, the computational techniques leverage one or more vitals, to determine the level of lifestyle discipline adherence and the level of Syndrome Specific adherence. The one or more vitals may correspond to, but not limited to, Triglyceride, Systolic Blood Pressure, Diastolic Blood Pressure, low-density lipoprotein cholesterol (LDL), high-density lipoprotein cholesterol (HDL), Body Weight, Heart Rate, Calories burned/day, and Alanine Transaminase (ALT).

In an example, the aforementioned vitals are non-limiting, and other examples of vitals may also be monitored to determine the adherence level of the patients corresponding to the test adherence, the drug adherence, the lifestyle discipline adherence, and the Syndrome Specific adherence. For example, different vitals may be monitored corresponding to different clinical clusters. For a patient in a clinical cluster corresponding to cancer vitals corresponding to cancer will be analyzed.

Furthermore, for assessment of whether the Syndrome Specific adherence (for example, diabetes is in control) is different for the patients in each of the clinical clusters. In an example, the management server 204 or 302 performs a diabetes in control check computation based on Triglyceride, Systolic Blood Pressure, Diastolic Blood Pressure, the LDL, Spot Microalbumin, and a patient risk profile including information regarding the risk level of the patient.

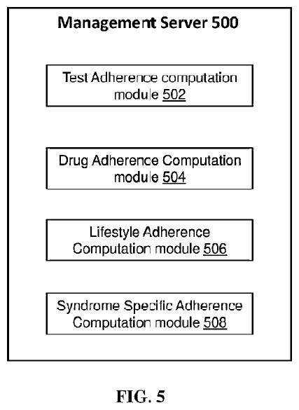

Now an explanation of the computation of the adherence score of the one or more patients into one or more patient clusters by the management server 204 or 302 will be made with reference to FIGS. 5, 6, 7A, 7B, and 8 of the drawings.

FIG. 5 illustrates a block diagram of adherence score computation modules of the of the management server of FIGS. 2 and 3, according to an embodiment of the present disclosure. FIG. 5 illustrates a management server 500 that includes a Test Adherence computation module 502, a Drug Adherence Computation module 504, a Lifestyle Adherence Computation module 506, Syndrome specific Adherence Computation module 508. Each of the adherence computation module is communicatively coupled with each other and can share the information generated as an output of the computation technique between each other. The management server 500 of FIG. 5 is same as the management server 204 and the management server 302 of FIGS. 2 and 3, respectively.

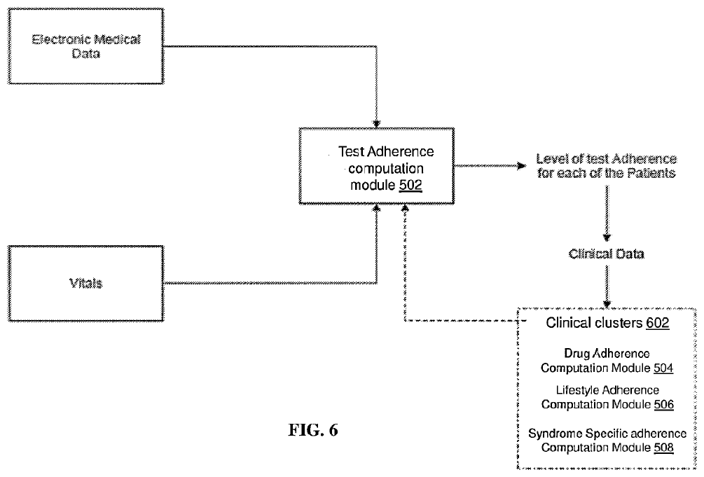

Referring now to FIG. 6, FIG. 6 illustrates an implementation of a test adherence computation technique for determining a level of adherence to tests for the one or more patients, according to an embodiment of the present disclosure. FIG. 6 illustrates the implementation of the test Adherence computation module 502 of FIG. 5 for determining the level of adherence to tests for the one or more patients.

As shown in the FIG. 6, the Test Adherence computation module 502 computes the test adherence score based on the electronic medical data of the patients in each of the clinical clusters 602 and the associated vital information corresponding to the patients in each of the clinical clusters 602. The clinical clusters 602 corresponds to the multiple clusters 406, 408, and 410 of FIG. 4. The computed test adherence score indicates a level of test adherence for each of the patients in the corresponding cluster among the clinical clusters 602. The computed test adherence score may serve as an indicator of an uptime of the patient device nodes 206. The Test Adherence computation module 502 may also share the computed test adherence score along with clinical data to the Drug Adherence Computation module 504, the Lifestyle Adherence Computation module 506, and the Syndrome specific Adherence Computation module 508.

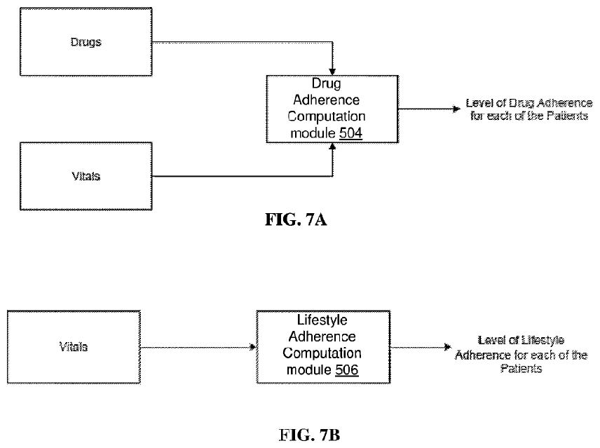

Referring now to FIG. 7A, FIG. 7A illustrates an implementation of a drug adherence computation technique for determining a level of adherence to drugs for the one or more patients, according to an embodiment of the present disclosure. Particularly, FIG. 7A illustrates the implementation of the Drug Adherence Computation module 504 of FIG. 5 for determining the level of adherence to the drugs for the one or more patients. The Drug Adherence Computation module 504 computes the drug adherence score based on drug information corresponding to the patients in each of the clinical clusters 602 and the associated vital information corresponding to the patients in each of the clinical clusters 602. The drug information may include one or more drugs prescribed to the corresponding patients in each of the clinical clusters 602. The computed drug adherence score indicates a level of drug adherence for each of the patients in the corresponding cluster among the clinical clusters 602.

Referring now to FIG. 7B, FIG. 7B illustrates an implementation of a lifestyle adherence computation technique for determining a level of adherence to lifestyle for the one or more patients, according to an embodiment of the present disclosure. Particularly, FIG. 7A illustrates the implementation of the Lifestyle Adherence Computation module 506 of FIG. 5 for determining the level of adherence to lifestyle for the one or more patients. The Lifestyle Adherence Computation module 506 computes the lifestyle adherence score based on the associated vital information corresponding to the patients in each of the clinical clusters 602. The associated vital information may include vitals related to lifestyle of the corresponding patients in each of the clinical clusters 602. The computed lifestyle adherence score indicates the level of adherence to lifestyle for each of the patients in the corresponding cluster among the clinical clusters 602.

Each of the computed drug adherence score and the lifestyle adherence score serve as an indicator of an extent to which each of a corresponding patient of the one or more patients works in cohesion with participants of the provider nodes 210 and the payor nodes 208. In a distributed network both the node up time and how effectively the node collaborates with the other nodes is critical for the healthcare network to be effective. Accordingly, the each of the computed test adherence score, the drug adherence score, and the lifestyle adherence score contributes together to make the healthcare network effective.

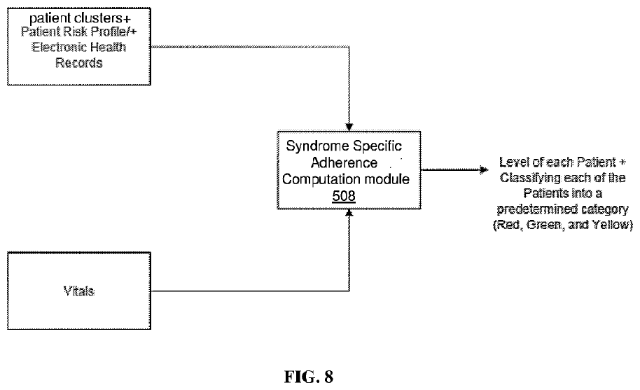

Referring now to FIG. 8, FIG. 8 illustrates an implementation an implementation of a syndrome specific adherence computation technique for determining a syndrome specific control level for one or more patients, according to an embodiment of the present disclosure. Particularly, FIG. 8 illustrates the implementation of the Syndrome Specific Adherence Computation module 508 of the FIG. 5 for determining the syndrome specific control level for the one or more patients. The Syndrome Specific Adherence Computation module 508 computes the Syndrome Specific Adherence score based on the patient risk profiles of the patients, the EHR of corresponding patients of the corresponding cluster among the clinical clusters 602, and cluster ID corresponding to each of the clinical clusters 602. The cluster ID indicates a specific clinical cluster of the patients among the clinical clusters 602. The computed Syndrome Specific Adherence score indicates the syndrome specific control level for each of the patients in the corresponding cluster among the clinical clusters 602. The Syndrome Specific Adherence Computation module 508 may also classifies the one or more patients into predetermined categories (for example, RED, GREEN and YELLOW) that indicates the risk profiles of the corresponding patients of the corresponding cluster among the clinical clusters 602.

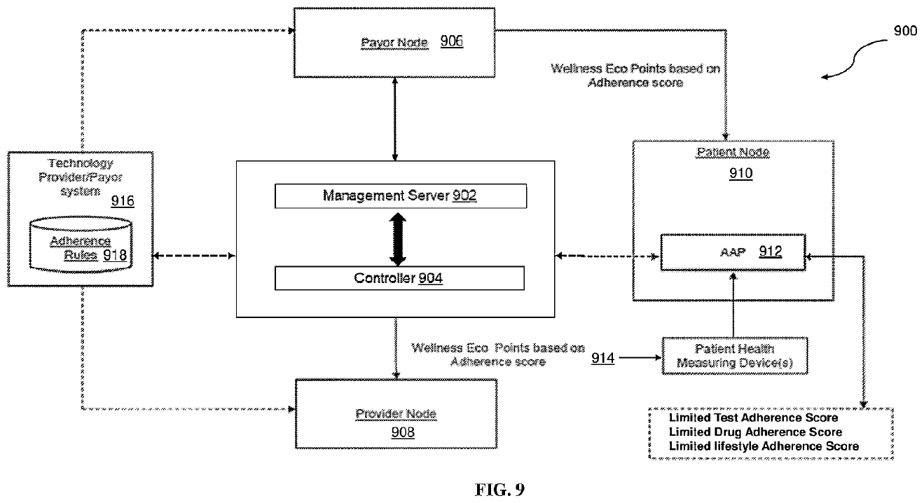

Referring back again to FIGS. 2 and 3, each of the patient device nodes 206, for example, but not limited to, the AAP device 308, further calculates a limited adherence score corresponding to each patient of the one or more patients at specific time intervals on daily basis. The calculated limited adherence score corresponds to at least one of a limited test adherence score, a limited drug adherence score, and a limited lifestyle adherence score corresponding to each patient in each of the one or more patient clusters. The calculation of the limited adherence score will be explained in detail with reference to the AAP device 912 of FIG. 9 of the drawings.

The patient device nodes 206 may also update criteria associated with parameters for the calculation of the limited adherence score specific to at least one cluster of the one or more clusters and at least one risk profile of a patient, based on a change in adherence parameters and any of criteria corresponding to the adherence parameters in platform services associated with one of the provider nodes 210 or the payor nodes 208.

The controller 304 validates an accuracy of limited adherence score calculated by each of the patient device nodes 206 based on a comparison of the calculated limited adherence score with the adherence score computed by the management server 204 or 302.

According to an embodiment of the present disclosure, now a detailed explanation of other functionalities of the management server 204 or 302, the controller 304, the patient device nodes 206 will be made using another example implementation of the blockchain based healthcare network 202 with reference to the FIG. 9 of the drawings.

FIG. 9 illustrates an example blockchain network 900 implementing a management server 902, a controller 904, a payor node 906, a provider node 908, a patient node 910 including an AAP Device 912, a patient health measuring device 914, and a technology provider and payor system 916, according to an embodiment of the present disclosure. Each of the individual nodes, systems, and devices of the blockchain network may have an Application Programming Interfaces (APIs) to communicate with each other.

The management server 902 is same as the management server 204 or 302. The payor node 906 corresponds to one of a payor node among the payor nodes 208. The provider node 908 corresponds to one of a provider node among the provider nodes 210. The patient node 910 including the AAP Device 912 corresponds to one of a patient device node among the patient device nodes 206. The AAP Device 912 is same as the AAP device of FIG. 3 and performs similar functions. Accordingly, it should be noted that in this embodiment, the components of the blockchain network 900, the components of the blockchain based healthcare system environment 200, and the components of the blockchain based healthcare system 300 which have the same name or functionality as those described above, the corresponding explanation will be omitted thereof.

The key actors in the healthcare network are the patients. The patients use medical devices for example but not limited to Continuous Glucose Meter (CGM), Blood Pressure Monitor (BPM) etc. . . . to measure blood sugar and blood pressure levels, several times in a day. The patient heath measuring devices 914 of the blockchain network 900 corresponds to such medical devices used by the patients. The AAP device 912 may also be provided to the patients.

The AAP device 912 measure the vitals based on the output of the patient heath measuring devices 914 and write the measured vitals directly into the patient's Electronic Medical Record (EMR) based on an approval of a digital consent by the patient in response to a requested consent by the AAP device 912. After the approval of the digital consent by the patient, the AAP device 912 modifies an access control list and the transaction is stored in the blockchain.

As an example, whenever the patient takes the blood sugar and blood pressure levels using the patient heath measuring devices 914, the AAP device 912 processes the calculated limited adherence score corresponding to at least one of the limited test adherence score, the limited drug adherence score, and the limited lifestyle adherence score of the patient as well as store the limited adherence score for a specific time period in a storage device, for example, but not limited to, the distributed immutable file system 216.

Further, the AAP device 912 the stored limited adherence score to the management server 902 as a batch process. Here, the calculation of the limited adherence score based on the vitals measurable using the heath measuring devices 914 is only illustrated as an example, in a non-limiting manner. The AAP device 912 or 308 may calculate limited adherence score based on other healthcare parameters associated with the patients.

The vitals measured by the heath measuring devices 914 may also be stored in a database hosted by the technology provider and payor system 916. As an example, in conjunction with the electronic medical record data elements like HbA1c, the LDL is then processed by the AAP device 912 to arrive at the limited adherence score of the patient as of a specific or particular day. The process of calculating the limited adherence score repeats every day to deliver the cumulative limited test adherence score, limited drug adherence score, limited lifestyle discipline adherence score, and syndrome specific adherence scores (like diabetes in control).

The technology provider and the payor system 916 are associated with the payor node 906 and the provider node 908 to provide various healthcare platform services. The technology provider and the payor system 916 sets adherence rules and the criteria of the adherence rules corresponding to the various healthcare platform services. The technology provider and the payor system 916 may set the adherence rules and the criteria of the adherence rules for a corresponding patient of one or more patients based on a type of the corresponding healthcare platform services.

Each of the patient node 910 including the AAP device 912 generates patient data items based on at least one of the vital information, a time stamp of measurement of the vitals, a patient id, and an adherence of a corresponding patient of the one or more patients towards the adherence parameters and criteria of the adherence parameters in platform services associated with one of the provider node 908 or the payor node 906. As an example, the AAP device 912 generates a hash based on the vitals measured by health measuring devices 914, the time stamp of the measurement, the calculated limited adherence scores, the adherence rules 918, and the patient ID. The AAP device 912 feeds the vitals with a time stamp into limited adherence logic pertaining to the test adherence, the drug adherence and the lifestyle discipline adherence in order to derive the limited adherence scores, and thus ensure trust worthiness of the patient-node 910.

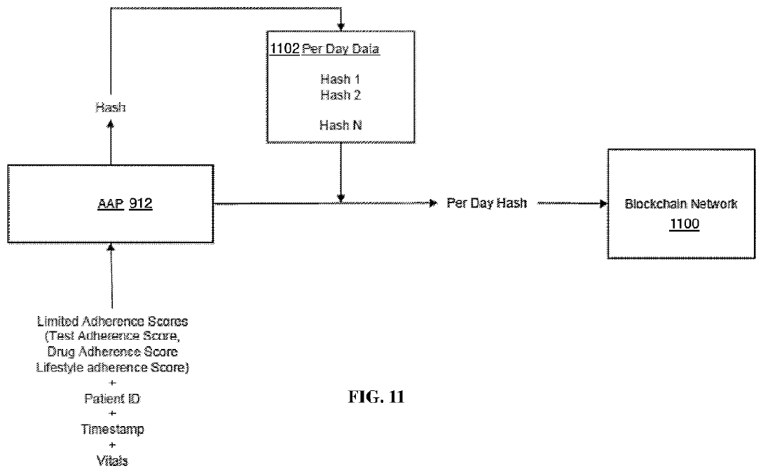

An example implementation of the generation of the hash and per day data including per day generated hash is shown with reference to FIG. 11 of the drawings. FIG. 11 illustrates an example implementation of the generation of hashes by the AAP device 912 and interaction with a blockchain network 1100, according to an embodiment of the present disclosure. The AAP device 912 generates the hash based on the vitals measured by heath measuring devices 914, the time stamp of the measurement, the calculated limited adherence scores, the adherence rules 918, and the patient ID and accordingly the per day data 1102 including per day hash (for example, Hash 1, Hash 2, . . . , Hash N) is generated.

In an example, the hash will be generated every time the vital is measured by the heath measuring devices 914. Furthermore, the AAP device 912 may also set a time parameter to generate a day hash based on the hashes generated during the day. For example, the day hash may be generated at 12:00 AM (typically all vital measurements for the day are done by 11:00 PM) on daily basis. The AAP device 912 further stores the per day data 1102 (per day hash) in the blockchain network 1100 to uniquely identify the transaction. The per day hash serves to connect the at least two devices involved viz one of the health measuring devices 914 and the AAP device 912 as well as to ensure the immutability of each of the vitals measurement.

The generation of the hash and the day hash helps building in an additional layer of trust. More particularly, it means that nodes other than the patient node in the blockchain based healthcare network 200 need not validate the data committed to the block by the patient node 910, and thus saves time and computation power. For example, considering a scenario where the patient uses a faulty blood sugar monitor and a blood pressure monitor to fake normal values. This will not be in the interest of the patient as the patient will not get the right medical counsel and this will in turn affect the quality of life of the patient. Further, the adherence scores calculated by the management server 204 or 302 involves vitals beyond the blood sugar and blood pressure for example but not limited to HbA1c. So, if a patient has to fake the normal values, the patient will have to fake 4 times every day and 365 days a year. Essentially the limited adherence scores at the patient node 910 is calculated every 4 hours during daytime and the adherence scores is calculated every day by the management server 204 or 302 at the hub helps to cross validate the adherence scores so that the patient cannot fake the normal values, and accordingly a right healthcare resource can be mapped for the patient, and this will in turn improve the quality of life of the patient.

Essentially the adherence computation techniques and limited adherence computation techniques contributes to improvement in the blockchain such that a patient need not be validated by other nodes of the blockchain network while saving the computation power and computation time and helps in the determination whether a specific syndrome the patient is suffering of is in control or not. The adherence computation techniques and the limited adherence computation techniques also helps in assessing the risk levels of the patient moving to a cluster of higher severity, thereby reducing the chances of the patient having co-morbidities.

According to an embodiment of the present disclosure, the AAP device 912 can operate in two modes that is a normal mode and an advance mode. The patients who are in clinical clusters with no co-morbidity can set the normal mode and the patients who are in clinical clusters with co-morbidities will be required to set the AAP device 912 in the advanced mode, which would mean the steps as described above will be initiated.

The AAP device 912 generates only one patient data item at a specific time on daily basis in a case if the AAP device 912 is set in a normal mode and generates multiple patient data items at multiple time intervals on the daily basis in a case if the AAP device 912 is set in an advance mode. As an example, for a patient whose AAP device 912 is in normal mode there will be only one hash (equivalent to the day hash) generated at 12:00 AM based on the vitals from the heath measuring devices 914, the time stamp of the measurement, the calculated limited adherence scores, and the patient ID.

Further, the AAP device 912 includes indicators for example but not limited to, based on diabetes in control levels, lifestyle discipline levels, wherein the AAP device 912 controls the indicators to indicate a need or requirement for the patient to have a teleconsultation with a physician and a peer coach respectively. The aforementioned AAP device AAP device 912 serves as a critical element to get patient node level data and associated adherence levels, directly from clinical devices after processing, which is critical for population health management especially in the context of communicable diseases.

The patient node 910 receives the allocated wellness Eco points from the management server 902 which is generated by payor nodes 208. The payor node 906 has the same functionalities as that of the payor nodes 208 of FIG. 2. The payor node 906 may also distribute the wellness Eco points to various actors in the healthcare ecosystem for example, but not limited to, Provider (Physician, Care-coordinator, Peer coach), Pharmacy, App developers, and retailers who sell Wellness products via the the management server 902. The distribution of the wellness Eco points by the payor node 906 is captured as transactions in the blockchain network 900.

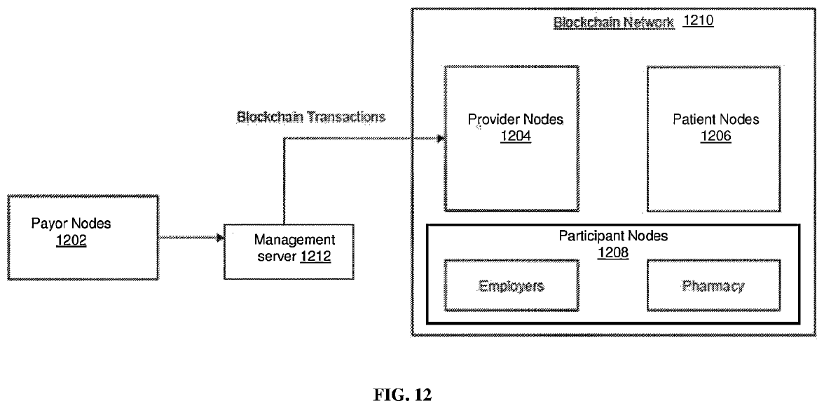

An example representation of the distribution of the wellness Eco points by the payor node 906 will be explained with reference to FIG. 12 of the drawings. FIG. 12 illustrates interactions between a payor node and a blockchain network, according to an embodiment of the present disclosure. FIG. 12 illustrates payor nodes 1202 and a blockchain network 1210 including provider nodes 1204, patient nodes 1206, participant nodes 1208, and a management server 1212. The participant nodes 1208 correspond, but not limited to, employers and pharmacy. The aforesaid example of the participant nodes 1208 is only illustrated for the purpose of example illustration and not limited in scope. The participant nodes 1208 may corresponds to any nodes that participate in the healthcare network.

As shown in FIG. 12, the payor nodes 1202 distributes the wellness Eco points (wellness points) to the various actors (provider nodes 1204, patient nodes 1206, and participant nodes 1208) of the blockchain network 1210 via the management server 1212. Thereafter, the distribution of the wellness Eco points by the payor nodes 1202 is captured as transactions in the blockchain network 1210. In particular, the wellness Eco points is distributed to the patient nodes 1206 by the payor nodes 1202 via the management server 1212 and further the management server 1212 distributes the received wellness Eco points to the provider nodes 1204 and the participant nodes 1208.

Referring back again to FIGS. 2, 3, and 9, the management server 204, 302, or 902 distributes the allocated wellness Eco points of the respective patient device nodes 206 to the provider nodes 210 and a respective participant node of the participant nodes 214 in the blockchain based healthcare network 202. As an example, the management server 904 distributes the wellness Eco points from the payor node 906 to the provider node 908 and respective participant nodes in the blockchain network 900.

Further with reference to FIGS. 3 and 9, the controller 304 or 904 generates a blockboard based on a ratio of number of the allocated wellness Eco points to a maximum number of wellness points that can be gained by the respective participant node of the participant nodes 214 or the participants nodes in the blockchain network 900. The ratio of the number of wellness Eco points gained to the maximum number of wellness Eco points that can be gained by the Provider nodes 210 or the provider node 908, the participant nodes 214 or the participant nodes in the blockchain network 900 are captured in the generated blockboard. The controller 304 may also controls the display screen of the output device 304 to display an interface of the generated blockboard. The blockboard serves as an indicator for the payor nodes 208 or the payor node 906 to diagnose the blockchain based healthcare network 200 or the blockchain network 900 across multi-level hierarchy and further includes specific action points corresponding to each of the one or more patients such that an effectiveness of vital measurement is improved.

Now, the interface of the generated blockboard will be explained in detail with reference to the FIG. 13 of the drawings. FIG. 13 illustrates a graphical user interface of a blockboard 1300 in a healthcare network platform, according to an embodiment of the present disclosure. As shown in FIG. 13, the graphical user interface of the blockboard 1300 includes at least one of a list of providers (Provider 1, Provider 2, Provider 3, and Provider 4), a list of physicians, a list of healthcare coordinators, a list of peer coach, and other participants (e.g. pharmacy, retailers of health products) in the blockchain based healthcare network 200 or the blockchain network 900 along with corresponding ratings. Further, as shown in FIG. 13, the graphical user interface of the blockboard 1300 also includes a summary of patterns (as shown in small black and white rectangular blocks corresponding to the physician, care coordinator, and peer coach in FIG. 13) associated with transactions across the blockchain based healthcare network 200 or the blockchain network 900, at least one cluster of the plurality of clusters (cluster name 1, cluster name 2, . . . , cluster name 8), and a plurality of selection icon related to a provider type (primary, secondary, tertiary), a risk level of the at least one cluster (low or high), adherence factors (for e.g. Life Style Discipline (LSD), Drug Adherence (DA), Diabetes In Control (DIC), Test Adherence (TA)), and a list of patients at risk including categorization of the risk levels (low, medium, high).

The summary of patterns is generated based on a selection of Adherence factor as the KPI to diagnose the blockchain network 900. The generation of summary of patterns is based on the ratio of the number of the allocated wellness Eco points to the maximum number of wellness points corresponding to the selected adherence factor for a corresponding blockchain transactions in the the blockchain network 900.

Using the graphical interface of the blockboard 1300, the management server 204 may also recommend at least one provider node among the provider nodes 210 from whom at least one patient in a specific clinical cluster is moved out to other provider nodes among the provider nodes 210 in the blockchain based healthcare network 200 based on a determination that the computed adherence score is less than a target level set by the payor nodes 208. As an example, once the regulator or the insurance company has diagnosed the network using the graphical interface of the blockboard 1300, the graphical interface of the blockboard 1300 has the facility to recommend the providers from whom the patients by cluster, who have to be moved out to other providers in the network, because of the fact that test adherence scores or drug adherence scores or life style discipline scores or diabetes in control scores of such patients are not at the target levels set by the regulator or the insurance company. These recommendations are essentially governed by various algorithms that combines the target levels set for each of the adherence factors and any other KPIs.

Using the graphical interface of the blockboard 1300, based on the computed adherence score the management server 204 may also recommend the risk profile of patients, patient's levels of adherence with adherence parameters and criteria of the adherence parameters, and a specific cluster of the plurality of clusters along with indication of a risk profile of patients in the specific cluster and at least one resource among one or more resources to at least one patient device node among the patient device nodes 206 for improvement in the adherence score. As an example, the management server 204 recommends the tests, drugs, educational video content, apps that the physician could prescribe, keeping in perspective their clinical clusters, the risk profile, and the adherence levels.

Using the graphical interface of the blockboard 1300, the management server 204 may also recommend the provider nodes 210 that which patients need to be moved to another clinical cluster based on adherence factor acceptable levels, and operational parameters specific to the provider nodes 210. This ensures the right patients are mapped to the right providers in the healthcare network and also ensures optimal utilization of resources. This will also help the physician to be proactive and help avert the movement to or increase in risk levels associated with co-morbidities. All of these transactions are captured as blockchain transactions and can be traced back to the patient vital levels to ensure auditability and disparities in the utilization of resources especially when it comes to a pandemic or epidemic situation. The management server 204 may also send alerts to a set of device nodes among the patient device nodes 206 belonging to the specific cluster along with the specific actions based on a determination that the specific cluster has patients with a high-risk profile.

In an example, the graphical interface of the blockboard 1300 helps the payor nodes 208 or the payor node 906 to diagnose the network across the multi-level hierarchy in order to determine the specific action points to increase the effectiveness of syndrome specific care to ensure that the patients do not develop co-morbidities.

The graphical interface of the blockboard 1300 can also provide ability to the payor nodes payor nodes 208 or the payor node 906 to diagnose the blockchain based healthcare network 200 or the blockchain network 900 based on a multitude of parameters and their combination—for example, but not limited to, diabetes in control levels, adherence factors, actors in the healthcare ecosystem, patient clusters, risk profile of the patients, providers based on a geographic location, types of the provider, and hence can arrive at specific deductions such that the patients do not develop co-morbidities.

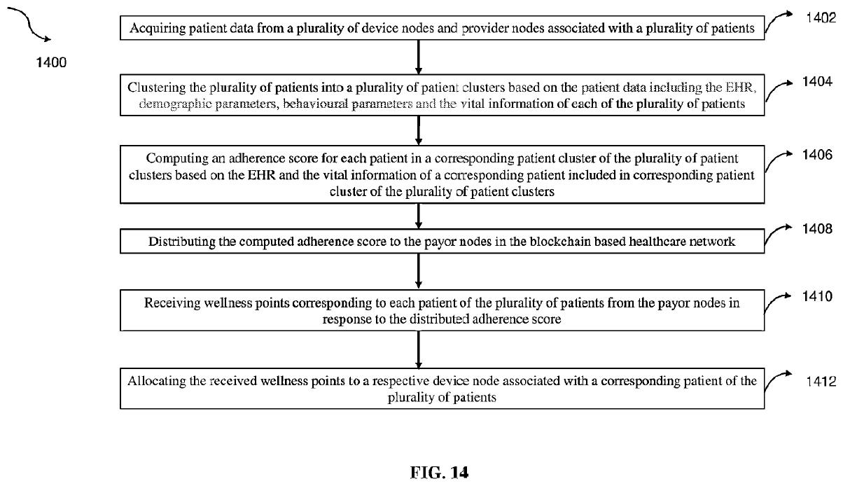

Now a flow chart of the method steps that can be performed the management server 204, 302, or 902 will be described with reference to FIG. 14 of the drawings. FIG. 14 illustrates the flow chart of the method steps performed by the management server 204, 302, or 902, according to an embodiment of the present disclosure. Since the functionalities of each of the management servers 204, 302, or 902 are same, for the ease of explanation the functionalities will be described with respect to the management server 204 only. Particularly, FIG. 14 illustrates a method 1400 for optimized utilization of resources in the blockchain based healthcare network 202.

The method 1400 comprises acquiring (step 1402) patient data from a plurality of device nodes and provider nodes associated with a plurality of patients. In the step 1402, the management server 204 acquires the patient data from the patient device nodes 206 and the provider nodes 210 associated with the one or more patients. The flow of the method 1400 now proceeds to (step 1404).

At the step 1404, subsequent to the acquisition of the patient data, the method 1400 comprises clustering the plurality of patients into a plurality of patient clusters based on the patient data including the EHR, demographic parameters, behavioural parameters and the vital information of each of the plurality of patients. In the step 1404, the management server 204 clusters the one or more patients into the one or more patient clusters based on the patient data including the EHR, the demographic parameters, the behavioral parameters, and the vital information of each of the one or more patients. The flow of the method 1400 now proceeds to (step 1406).

At the step 1404, subsequent to the clustering of the one or more patients, the method 1400 comprises computing an adherence score for each patient in a corresponding patient cluster of the plurality of patient clusters based on the EHR and the vital information of a corresponding patient included in corresponding patient cluster of the plurality of patient clusters. In the step 1406, the management server 204 computes an adherence score associated with the at least one KPI for each patient in the corresponding patient cluster of the one or more patient clusters based on the EHR and the vital information of the corresponding patient included in the corresponding patient cluster of the one or more patient clusters. The flow of the method 1400 now proceeds to (step 1408).

At the step 1404, subsequent to the computing of the adherence score, the method 1400 comprises distributing the computed adherence score to the payor nodes in the blockchain based healthcare network. In the step 1404, the management server 204 distributes the computed adherence score to the payor nodes 208. The management server 204 may also distribute the computed adherence score to the provider nodes 210. The flow of the method 1400 now proceeds to (step 1410).

At the step 1410, subsequent to the distribution of the computed adherence score, the method 1400 comprises receiving wellness points corresponding to each patient of the plurality of patients from the payor nodes 208 in response to the distributed adherence score. In the step 1410, the management server 204 receives the wellness Eco points transmitted by the payor nodes 208. The flow of the method 1400 now proceeds to (step 1412).

At the step 1412, subsequent to the reception of the wellness points, the method 1400 comprises allocating the received wellness points to a respective device node associated with a corresponding patient of the plurality of patients. In the step 1412, the management server 204 allocates the received wellness Eco points to each of the provider nodes 210 and a respective patient device node among the patient device nodes 206 associated with a corresponding patient of the one or more patients.

Now some other functionalities of the management server will be explained in accordance with another embodiment of the present disclosure.

In the case of healthcare network there are some other aspects as well different from those described above. One such example aspect is accountability of the patient. Human lives could be lost if the patient data that is collected by a wearable device is not transmitted to the physician on time especially in a medical emergency or the IoT gateway on the provider side is not functioning. As such it is important that the healthcare network participants can monitor that the APIs associated with applications and wearables, intelligent cameras that do patient surveillance and the medical devices, the IoT gateways and Internet gateways are transmitting data across the digital network. Every network participant should have access to a decentralized system that the APIs, the IoT gateways, and internet gateways can update whether they have transmitted the data on time and every network participant has a copy of this, that is immutable and trustworthy.

Such healthcare network may include multiple routes to connect with the patients. For example, a first route that connects the patient to the hospital/provider and then to the regulator or the payor (referred to as aggregator) and a second route that connect the patient directly to the aggregator. In both cases the telecommunication entity may act as the intermediaries. The wearables devices, the applications/systems, the IoT gateway and the internet gateways can serve as intermodal modes in the healthcare network responsible for transporting the data. In such type of healthcare network, the management server 204 may perform a series of step to ensure the accountability of the patients. Initially, the management server 204 computes aggregation points for each of nodes in the healthcare network in a specific route for at least one provider node among the provider nodes 210. The specific route corresponds to one of the first route or the second route. The aggregation points are computed by the management server based on a reception and transmission events of data among the source nodes, the destination nodes, and the intermediate nodes of the healthcare network. For the computation of the aggregation points firstly, the intermediate nodes are given the greatest number of system aggregation points and are a function of the source and destination node. Secondly, response to a transmission request is taken into account for the network to work and is a function of a number of branches the data has to be transmitted from one node to another node. The management server 204 may use a multiplier in a case the aggregator requests to increase the importance of the uptime of the intermediate nodes.

Subsequent to the computation of the aggregation points, the management server 204 determines whether any response is received from at least one of the APIs, the IoT Gateway, or the Internet gateway for a specific service. Further, the management server 204 computes a node performance factor (NPF) of each of the nodes in the healthcare network based on the computed aggregation points and a result of the determination regarding the reception of the response from the least one of the APIs, the IoT Gateway, or the Internet gateway for the specific service. The management server 204 may also compute an Aggregated Node Performance Factor (ANPF) by multiplying the computed node performance factor with the computed aggregation points. The NPF and the ANPF are used as additional mechanism for authenticating the nodes in the blockchain network. While NPF is used as the mechanism to check on the reliability of the node in transferring data, ANPF is used as the mechanism to order the transactions in a block.

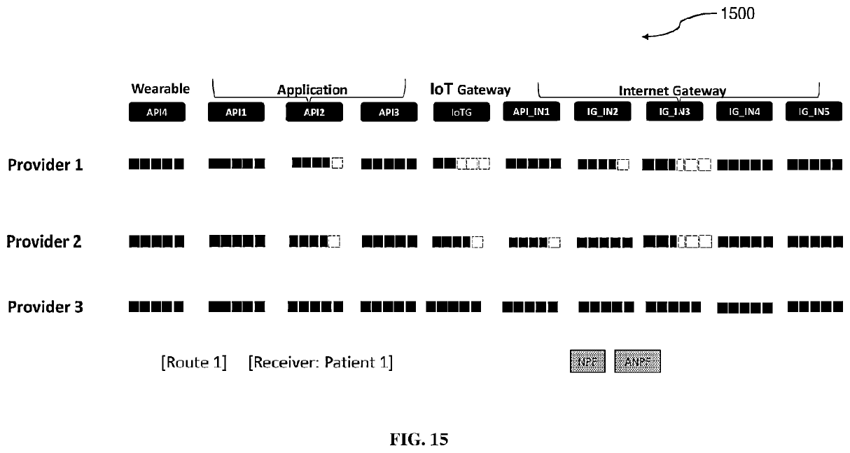

Further, the controller 304 or 904 may also generate a graphical user interface in a form of the blockboard for each of the first route and the second route. An example of the graphical user interface in the form of the blockboard is shown in FIG. 15 of the drawings. FIG. 15 illustrates an example of the graphical user interface in the form of the blockboard for the first route, according to another embodiment of the present disclosure. FIG. 15 depicts the blockboard 1500 which indicates how the network of each provider along the first route is performing. As shown in FIG. 15, 5 blocks under a node, denote an NPF of 100%. From the blockboard 1500 it is evident that the Hospital 1 is the worst performing and it is primarily the IoT gateway that is not reliable and has to be rectified. Further, it is evident that Hospital 2 has to closely monitor their IoT Gateway. Internet gateway IG_IN3 from Telco 2 is not reliable and will need to have redundancy built-in. Furthermore, it is evident that Hospital 3's network is very reliable, and the payor should look at putting their high-risk patients in Hospital 3's network, assuming all the 3 providers are equally capable from a clinical standpoint.

Furthermore, the management server may also determine whether a value of the computed NPF is less than a specific threshold level of the NPF, and further eliminates a set of nodes in the healthcare network of which the NPF is less than the specific threshold level based on the determination. The specific threshold level of the NPF can be set by the aggregator based on a specialty, cluster, and a risk level of a patient in the clinical cluster. This ensures that only those nodes which are reliable in transferring data (indicated by the NPF) and contribute significantly in serving the patient with respect to data transfer (indicated by the ANPF) are allowed to do transactions in the blockchain network. Those nodes that do not meet the specific threshold level of the NPF are automatically eliminated by the management server 204 from the network until they improve the response from the concerned wearable API, application API, internet gateway, or the IoT gateway. Due to the elimination of such nodes the computing power required across the network can be reduced, means there are a lesser number of nodes participating in the consensus mechanism in the blockchain. Further, the use of the NPF and the ANPF serves as a basis for selecting validators thereby reducing the level of centralization. Furthermore, the elimination of the nodes helps in reduction of the computing power required across the blockchain network. Using the NPF and ANPF additional authenticating mechanisms the security of the blockchain will also be enhanced. Using the above-described healthcare network and its functionalities, the aggregator can identify high risk patients with a hospital that has a high NPF, thereby reducing the chances of medical emergency due to non-availability of patient data on time. The proposed methods using the NPF and ANPF contributes to evaluation of every transmission of data by the APIs/IoT gateway/Internet gateway for response in an immutable trust-worthy manner and thereby the patients in critical conditions can be monitored and human lives can be saved.

The proposed systems and methods may implement a game-based interface for the patients that can be driven based on cluster information, risk profile information, and the adherence computation techniques.

The proposed systems and methods of the present disclosure helps in driving healthcare network monitoring and provides optimum utilization of resources involved in the healthcare network. Furthermore, based on the proposed computation techniques, graphical user interface of the blockboard 1300, the payors can be able to assess, for example:

(a) Information regarding providers who are good at managing a type of cluster to ensure the incidence of comorbidities,

(b) level of focus required corresponding to one of the primary care, the secondary care, or the tertiary care, or

(c) Areas needs to focus on—for example, under care-ordination there is a need to focusing on test adherence and the drug adherence.

The proposed systems and methods of the present disclosure can help a provider in identifying specific actors or people to assess their performance. The proposed systems and methods of the present disclosure can help a payor in rewarding providers and in turn the healthcare actors based on the outcomes they contribute when it comes to the health of the patient—which in turn reduces the incidence of comorbidities (where the onus is more on the physician) and increases adherence levels (where the onus is more on the care-coordinator and pharmacy) and increases life style discipline (where the onus is more on the peer coach).

The aspects of the proposed systems and methods of the present disclosure described herein aids prognosis of co-morbidities and reduction in the cost of care and make healthcare affordable.

The proposed systems and methods of the present disclosure also contribute to Increasing the effectiveness of the physician consult and a digital assessment of the data that is an output from the AAP devices across the patient nodes. Using the proposed systems and methods, the physician can now focus on ascertaining if the recommendation is right, form a hypothesis about the patient and ask specific questions that will help the physician to arrive at the right medical device during teleconsultation.

The proposed systems and methods of the present disclosure can also help in increasing the patient to healthcare actor ratio digitally. Essentially, in addition to the network resource optimization, the span for the healthcare actor is also increased. The healthcare actors can now focus their attention to the patients that need attention which is critical for a non-communicable disease like diabetes, patients can get advice proactively from the healthcare actors, and the care groups which is a critical factor for diabetes care can be well informed in advance about the state of the patient. Using the proposed systems and methods of the present disclosure, the pharmacy which currently plays a passive role can also proactively reach out to the patients who are low on drug adherence using the blockboard interface.

The proposed systems and methods of the present disclosure can also help in increasing the members engagement in case of specific syndrome like diabetes which is life long and is a lifestyle disease in most cases. Engaging the members is critical to ensure that they do not have co-morbidities/keep it in control. To increase the member engagement, the proposed systems and methods of the present disclosure recommends products approved by the physicians of such patients. The recommended products here may include, but not limited to, educational video content that is of relevance to the members based on their clinical condition, healthy diet packages, and Wellness apps. Also, the wellness Eco points allocated by the proposed systems and methods can be uses by the patients or members to buy the products approved by the physician. More importantly, the Wellness Eco points here serves as a motivating factor as it helps the patients to gauge how well are they doing in response to the therapy without knowing the clinical intricacies.

1. A method for optimized utilization of resources in a blockchain based healthcare network, comprising:
acquiring, by a management server, patient data from a plurality of device nodes and provider nodes associated with a plurality of patients, wherein the patient data includes Electronic Health Record (EHR) and vital information of each of the plurality of patients;
clustering, by the management server, the plurality of patients into a plurality of patient clusters based on the patient data including the EHR, demographic parameters, behavioral parameters, and the vital information of each of the plurality of patients;
computing, by the management server, an adherence score associated with at least one Key Performance Indicator (KPI) for each patient in a corresponding patient cluster of the plurality of patient clusters based on the EHR and the vital information of a corresponding patient included in corresponding patient cluster of the plurality of patient clusters;
distributing, by the management server, the computed adherence score to payor nodes in the blockchain based healthcare network;
receiving, by the management server, wellness points corresponding to each patient of the plurality of patients from the payor nodes in response to the distributed adherence score, wherein the wellness points is based on the computed adherence score; and
allocating, by the management server, the received wellness points to the provider nodes and a respective device node associated with a corresponding patient of the plurality of patients.

2. The method as claimed in claim 1, further comprising:
calculating, by each of the plurality of device nodes, a limited adherence score corresponding to each patient of the plurality of patients at specific time intervals on daily basis; and
validating, by a controller of the blockchain based healthcare network, an accuracy of the calculated limited adherence score based on a comparison of the calculated limited adherence score with the adherence score computed by the management server.

3. The method as claimed in claim 2, further comprising updating, by at least one device node of the plurality of device nodes, criteria associated with parameters for the calculation of the limited adherence score specific to at least one cluster of the plurality of clusters and at least one risk profile of a patient, based on a change in adherence parameters and any of criteria corresponding to the adherence parameters in platform services associated with one of the provider nodes or the payor nodes.

4. The method as claimed in claim 2, further comprising:
distributing, by the management server, the allocated wellness points of the respective device node to respective participant node of a plurality of participant nodes in the blockchain based healthcare network; and
generating, by the controller, a blockboard based on a ratio of number of the allocated wellness points to a maximum number of wellness points that can be gained by the respective participant node of the plurality of participant nodes, wherein
the blockboard includes at least one of a list of providers, a list of physicians, a list of healthcare coordinators, and other participants in the blockchain based healthcare network along with corresponding ratings, and
the blockboard serves as an indicator for the payor nodes to diagnose the blockchain based healthcare network across multi-level hierarchy and further includes specific action points corresponding to each of the plurality of patients such that an effectiveness of vital measurement is improved.

5. The method as claimed in claim 4, wherein the blockboard further includes a summary of patterns associated with transactions across the blockchain based healthcare network, at least one cluster of the plurality of clusters, and a plurality of selection icon related to a provider type, a risk level of the at least one cluster, adherence factors, and a list of patients at risk.

6. The method as claimed in claim 1, wherein
the EHR includes at least one of vital information, clinical information, demographic information, and behavior information of the plurality of patients,
the computed adherence score corresponds to at least one of a test adherence score, a drug adherence score, a lifestyle adherence score, and a syndrome specific score corresponding to each patient in each of the plurality of patient clusters,
the test adherence score serves as an indicator of an uptime of the plurality of device nodes, and
the drug adherence score and the lifestyle adherence score serve as an indicator of an extent to which each of a corresponding patient of the plurality of patients works in cohesion with participants of the provider nodes and the payor nodes.

7. The method as claimed in claim 1, further comprising:
generating, by each of the plurality of device nodes, patient data items based on at least one of the vital information, a time stamp of measurement of vitals, a patient id, and an adherence of a corresponding patient of the plurality of patients towards adherence parameters and criteria of the adherence parameters in platform services associated with one of the provider nodes or the payor nodes; and
storing, by each of the plurality of device nodes, the generated patient data items in a database of the blockchain based healthcare network associated with one of the provider nodes or the payor nodes, wherein the adherence parameters and the criteria of the adherence parameters for a corresponding patient of the plurality of patients is set by one of a corresponding provider node among the provider nodes or a corresponding payor node among the payor nodes.

8. The method as claimed in claim 1, further comprising:
generating, by at least one first device node among the plurality of nodes, only one patient data item at a specific time on daily basis in a case if the at least one first device node is set in a normal mode; and
generating, by at least one second device node among the plurality of nodes, multiple patient data items at multiple time intervals on the daily basis in a case if the at least one second device node is set in an advance mode, wherein
the normal mode indicates a patient with no co-morbidity associated with a clinical cluster of the plurality of patient clusters, and
the advance mode indicates a patient with co-morbidity associated with the clinical cluster of the plurality of patient clusters.

9. The method as claimed in claim 1, further comprising:
moving, by the management server, at least one patient from a first cluster of the plurality of clusters to second cluster of the plurality of clusters based on a progression of the wellness points corresponding to the at least one patient;
recommending, by the management server, at least one provider node of the provider nodes from whom at least one patient in a specific cluster is moved out to other provider nodes in the blockchain based healthcare network, based on a determination that the adherence score is less than a target level set by the payor nodes;
recommending, by the management server based on the computed adherence score, the risk profile of patients, and patient's levels of adherence with adherence parameters and criteria of the adherence parameters, a specific cluster of the plurality of clusters along with indication of a risk profile of patients in the specific cluster and at least one resource among a plurality of resources to at least one device node for improvement in the adherence score; and
sending, by the management server, alerts to a set of device nodes among the plurality of device nodes belonging to the specific cluster along with specific actions based on a determination that the specific cluster includes patients having a high-risk profile.

10. The method as claimed in claim 1, further comprising:
computing, by the management server, aggregation points for each of nodes in the blockchain based healthcare network in a specific route for at least one provider node;
determining, by the management server, whether any response is received from at least one of an Application Programming Interface (API), Internet of Things (IoT) Gateway, or Internet gateway in the blockchain based health care network for a specific service;
computing, by the management server, a node performance factor of each of the nodes based on a result of the determination and the computed aggregation points;
determining, by the management server, whether a value of the computed node performance factor is less than a specific threshold level of the node performance factor; and
eliminating, by the management server, a set of nodes of which the node performance factor is less than the specific threshold level based on the determination.

11. The method as claimed in claim 1, further comprising:
acquiring, by a patient debug device in the blockchain based healthcare network, a plurality of criteria of each of adherence parameters corresponding to the computed adherence score;
displaying, on a display screen of the patient debug device based on the acquired plurality of criteria, a deviation chart including information regarding at least one of a positive deviation and a negative deviation of a corresponding patient of the plurality of patients against each of the plurality of criteria with respect to each of a baseline value and a threshold value for each of the plurality of criteria; and
generating an adherence snapshot of adherence rules based on user's input against the plurality of criteria with reference to the information included in the displayed deviation chart, wherein
the generated adherence snapshot is auditable and editable based on the user's input, and
a pattern of the adherence snapshot is generated as a function of time in response to corresponding user inputs against the plurality of criteria.

12. A system for optimized utilization of resources, comprising:
a plurality of device nodes and provider nodes associated with a plurality of patients, wherein the plurality of device nodes and provider nodes are configured to generate patient data that includes Electronic Health Record (EHR) and vital information of each of the plurality of patients;
a plurality of payor nodes; and
a management server configured to:
acquire the patient data from the plurality of device nodes and the provider nodes associated with the plurality of patients;
cluster the plurality of patients into a plurality of patient clusters based on the patient data including the EHR, demographic parameters, behavioral parameters, and the vital information of each of the plurality of patients;
compute an adherence score associated with at least one Key Performance Indicator (KPI) for each patient in a corresponding patient cluster of the plurality of patient clusters based on the EHR and the vital information of a corresponding patient included in corresponding patient cluster of the plurality of patient clusters; and
distribute the computed adherence score to the plurality of payor nodes, wherein the plurality of payor nodes is configured to:
generate wellness points corresponding to each patient of the plurality of patients based on the computed adherence score; and
transmit the generated wellness points to the management server in response to the distributed adherence score;
receive the wellness points from the plurality of payor nodes; and
allocate the received wellness points to the provider nodes and a respective device node associated with a corresponding patient of the plurality of patients.

13. The system as claimed in claim 12, further comprising a controller, wherein
each of the plurality of device nodes is configured to calculate a limited adherence score corresponding to each patient of the plurality of patients at specific time intervals on daily basis, and
the controller is configured to validate an accuracy of the calculated limited adherence score based on a comparison of the calculated limited adherence score with the adherence score computed by the management server.

14. The system as claimed in claim 13, further comprising a plurality of participant nodes, wherein
the management server is further configured to distribute the allocated wellness points of the respective device node to a respective participant node of the plurality of participant nodes,
the controller is further configured to generate a blockboard based on a ratio of number of the allocated wellness points to a maximum number of wellness points that can be gained by the respective participant node of the plurality of participant nodes,
the blockboard includes at least one of a list of providers, a list of physicians, a list of healthcare coordinators, and other participants in the blockchain based healthcare network along with corresponding ratings, and
the blockboard serves as an indicator for the payor nodes to diagnose the blockchain based healthcare network across multi-level hierarchy and further includes specific action points corresponding to each of the plurality of patients such that an effectiveness of vital measurement is improved.

15. The system as claimed in claim 12, further comprising a plurality of nodes, an Application Programming Interface (API), an Internet of Things (IoT) Gateway, and an Internet gateway, wherein the management server is further configured to:
compute aggregation points for each of nodes of the plurality of nodes in a specific route for at least one provider node;
determine whether any response is received from at least one of the API, the IoT Gateway, or the Internet gateway for a specific service;
compute a node performance factor of each of the plurality of nodes based on a result of the determination and the computed aggregation points;
determine whether a value of the computed node performance factor is less than a specific threshold level of the node performance factor; and
eliminate a set of nodes among the plurality of nodes of which the node performance factor is less than the specific threshold level based on the determination.
